(19) United States
(12) Patent Application Publication
(10) Pub. No.: US 2012/0191041 A1
(43) Pub. Date: Jul. 26, 2012

(54) Safety syringe

(76) Inventor: Salvatore V. Pullara, Rome, NY (US)

(21) Appl. No.: 12/931,053

(22) Filed: Jan. 21, 2011

(51) Int. Cl. A61M 5/50

(52) U.S. Cl. 604/110

(57) ABSTRACT

An improved syringe comprises a tube; a resilient needle retainer releasably installed therein; a needle being threadably installed in the needle retainer; a break-away plunger; and a sealing means in the form of a multi-pleated bellows secured to the plunger using a retaining element. The break-away plunger may comprise a plunger with four flanges arranged into a cruciform-shaped cross-section, and with each flange further comprising a specially-shaped narrow cut to aid the breaking process, while leaving the plunger with more lateral stability during use. The multi-pleated bellows improves upon standard piston-plunger arrangements, as each of the bellows may seal independently upon an inside surface of the tube. The needle retainer is releasably installed in the tube for use during an injection using a detent, and is restrained relative to the tube, after retraction of the needle during breaking off of the plunger, using a second detent.

### FIELD OF THE INVENTION

The present invention relates to improvements in medical syringes, and more particularly to features to enhance the safety of syringes once they are used, and are then ready for disposal.

### BACKGROUND OF THE INVENTION

Hypodermic medical syringes are used to inject liquid or gasses below the skin of a person into body tissues. These syringes typically comprise a needle affixed to a tube, and a plunger and/or piston moving within the tube being usable to either draw in or expel the substance. The tube or barrel portion of the syringe had often been made of glass, to permit later sterilization in an autoclave for subsequent reuse. The popularity of manufacturing syringes using plastic materials increased dramatically because its cost-effectiveness permitted one-time use and disposal of the syringes thereafter. The desirability of a single use syringe is derived from the need to reduce the risk of spreading blood-borne diseases from repetitive usage. Of particular concern is the reduction of risk in spreading hepatitis and the HIV virus.

Another need with regard to the safety of hypodermic syringes arose from the occupational hazard within the medical community, and concerns accidental needle-stick injuries to medical professionals, health care workers, nurses, surgeons, etc. Where a needle has been used on a patient in drawing blood, administering an intramuscular or intravenous drug, or performing other procedures, and the needle slips from the caregivers hand and accidentally penetrates a glove and skin, the potential for the transmission of viruses from the patient to the caregiver poses a serious risk, especially when the patient is known to be infected. While the infectious nature of HIV and the hepatitis C virus (HCV) decrease within a couple of hours, the hepatitis B virus (HBV) remains stable even in dried blood for over a week. The frequency of accidental needle-stick injuries is estimated to be 800,000 per year in the United States and 3.5 million worldwide, with annual world-wide consequences of 66,000 HBV infections, 16,000 HCV infections, and 1,000 HIV infections.

So, proper syringe handling and disposal is a paramount concern. There have been many approaches to disposal. For instance, U.S. Pat. No. 4,315,448 to Ball for “Hypodermic Needle Destructor” discloses a method that is usable by the caregiver to immediately cut off the needle with a cam-actuated knife blade. The needle of the syringe is inserted into an opening in the center of a knob, which connects to a knife blade. The knob may be rotated to cause the blade to pivot against the base plate and shear off the portion of the needle extending through the opening, with the sheared needles being collected in a receptacle box. However, one of the drawbacks to this early disposal method is that a portion of the needle may still be protruding from the syringe, and may nonetheless potentially scrape a caregiver as he/she seeks to dispose of the remainder of the syringe.

U.S. Pat. No. 4,850,977 to Bayless discloses an early design for safely protecting the needle of a used syringe. The Bayless invention includes a slidable attachment on the end of the syringe, which is spring-loaded thereon, with a push-button lock keeping the sheath in an inactivated position. Once the syringe has been used to inject a patient, the care-giver may simply press the button and permit the sheath to be spring-driven to cover the needle. While the invention serves to protect the caregiver against accidental needle stick injuries, the extra parts necessary for construction of the device adds unnecessary complexity and expense, and also makes the syringe bulky.

An improvement is shown by U.S. Pat. No. 5,242,400 to Blake for a “Disposable Pre-Filled Syringe with Retractable Needle.” The Blake syringe comprises a plunger that supports a retractable needle being slidably disposed within a barrel. The plunger is restrained from exiting the barrel end that is distal from the needle, through the use of locking tabs that are integral to the barrel. The plunger shaft may be inserted into the barrel to be connected to the plunger so as to be able to drive it longitudinally within the barrel, as well as to be able to drive it to rotate relative to the barrel. Shearable tabs on the plunger shaft keep the shaft from being completely withdrawn from the barrel due to engagement with the plunger, and the plunger's engagement with the barrel's locking tabs. After using the syringe, the caregiver may drive the plunger further inward, using the plunger shaft, so that the locking wedges of the plunger contact catch locks on a needle hub receptacle, after which the plunger may be rotated for engagement therebetween. The plunger shaft may be withdrawn until the plunger contacts the locking tabs, with the needle then being disposed within the barrel. With the continued application of “a large enough retracting force,” the shear tabs on the plunger shaft break off and permit the plunger shaft to be disconnected from the plunger and removed from the syringe. While comprising an improvement over the Bayless syringe, the Blake syringe suffers from a couple of drawbacks. Requiring the caregiver to apply “a large enough retracting force” to cause the shear tabs to shear off poses a safety hazard, and furthermore mandates that every caregiver have the sufficient strength to be able to accomplish the task. Even where the caregiver possesses the requisite strength, which may not always be the case, requiring the care giver to unnaturally exert the two opposing forces to the barrel and the plunger shaft with each hand, will result in the rapid movement of those two parts away from each other when the tabs actually do shear off. While the user seeks to cease applying that force, either of the parts may accidentally strike an object or even a person, with the potential for a portion of the long needle to be flung out the end of the barrel and transmit any residual fluid contained thereon.

An improvement to the Blake syringe is found in U.S. Pat. No. 7,806,860 to Walton, and in a similar syringe in Walton's pending U.S. patent Application No. 2009/0171287. With those syringes, rather than needing to apply the large retracting force required with Blake's invention, a unitary plunger and shaft comprise a shaft with a “break point.” This break point comprises a reduced cross-sectional area that permits the user, after withdrawing the needle to be within the barrel, to snap off a portion of the shaft, wherein the flanges on the plunger are twisted through the twist lock sleeve fitted in the end of the barrel. However, the Walton syringes are both in name and in their functioning, single use syringes, because the locking head at the end of the plunger will automatically engage the fingers, as the plunger is depressed to completely inject the medicant. While a single use philosophy for syringes has become standard operating procedure in certain areas of the medical community, it is not routinely practiced, nor even practical, with respect to use in certain other communities. For example, it is common among the non-affluent, particularly in developing countries, for a disposable syringe to be used multiple times among diabetics for insulin injections. In a British Medical Journal article, this multiple use practice is found to generally be safe, with cross-infection not posing a problem when diabetes patients uses the same syringe only for self-injection (see: “Multiple use of Ordinary Disposable Syringes for Insulin Injections,” British Medical Journal, vol. 284, 23 January, 1983, available at www.ncbi.nlm.nih.gov/pmc/articles/PMC1495773/pdf/bmjcred00590-0022.pdf). For this, and other reasons, the syringe disclosed herein provides desired improvement over the prior syringes.

### OBJECTS OF THE INVENTION

It is an object of the invention to provide a syringe that may be suitably used as a single use syringe, and may also be used, when necessary, for multiple injections.

It is another object of the invention to provide a single use syringe that easily accommodates disposal by a care-giver, regardless of the caregiver's size and strength.

It is a further object of the invention to provide a single-use disposable syringe that may be quickly assembled in a simple manufacturing process.

It is another object of the invention to provide a single-use disposable syringe that may be easily withdrawn into the barrel after use, while being easily secured therein during and after destruction of the plunger shaft.

It is also an object of the invention to provide a plunger sealing means that maintains its sealing integrity during destruction of the plunger shaft.

Further objects and advantages of the invention will become apparent from the following description and claims, and from the accompanying drawings.

### SUMMARY OF THE INVENTION

A safety syringe of the present invention may comprise a tube; a resilient needle retainer being releasably installed in the tube; a needle with a portion of a base of the needle being threadably installed in the needle retainer; a break-away plunger; and a sealing means in the form of a multi-pleated bellows being secured to the plunger using a retainer in the form of a nut-like element.

The break-away plunger may comprise a plunger with a series of flanges protruding outward from an axis. As an example, in one embodiment there may be three flanges arranged to form a Y-shaped cross-section. In another embodiment, there may be four flanges arranged into a cruciform-shaped cross-section, which is typical of most plastic syringes. Each of the flanges may further comprise a narrow cut. In one embodiment, the narrow cut may begin at an edge of each of the flanges. In a second embodiment, each of the narrow cuts may instead begin and end away from a respective edge of each of the flanges, and to better facilitate the break-away feature of the plunger, each of the narrow cuts may begin and end with a V-shaped notch, with each of the V-shaped notches at the beginning of each cut connecting to a respective edge of each of the flanges using a hairline slit. This arrangement serves to aid the breaking process, while leaving the plunger with more lateral stability during use by the health care professional. The V-shaped notch at the end of each of the cuts may also connect to a beginning of an adjacent flange using another hairline slit.

The safety syringe herein may utilize a sealing means in the form of a multi-pleated bellows, which may preferably comprise three or more pleats, so that the three or more pleats may each seal independently upon an inside surface of the tube. The needle retainer of the safety syringe herein may be releasably installed in one end of the tube for use during an injection using a detent, with the detent comprising an annular bearing surface of the needle retainer being received in a corresponding recess in the tube. The tube of the safety syringe may be tapered, with the needle retainer being correspondingly tapered for engagement therebetween, so that the engagement works in combination with the detent for sealing, and in providing the releasable installation of the needle retainer in the tube. The needle retainer may also be retained within the tube at an opposite end during breaking off of the plunger using a second detent arrangement, which may comprise the same annular bearing surface protruding from the needle retainer being received by a second recess in the tube.

The breaking off of the plunger may begin with the securing of the plunger to the needle retainer by threadably engaging the plunger with the needle retainer. The threadable engagement may be by first pushing the plunger to contact needle retainer, and then by applying a torque to the plunger to cause the engagement. The plunger may then be used to retract the needle into the tube, until the needle retainer engages the second detent to secure it thereto. The user may then simply break off a portion of the plunger by applying a small bending force to the tube and the plunger.

### BRIEF DESCRIPTION OF THE DRAWINGS

FIG. 1 is an exploded view of the component parts of the safety syringe of the current invention.

FIG. 1A is a view of the needle of the safety syringe of FIG. 1.

FIG. 1B is a view of the tube of the safety syringe of FIG. 1.

FIG. 1C is a view of the needle retainer of the safety syringe of FIG. 1.

FIG. 1D is a view of the bellows retainer of the safety syringe of FIG. 1.

FIG. 1E is a view of the bellows of the safety syringe of FIG. 1.

FIG. 1F is a view of the plunger of the safety syringe of FIG. 1.

FIG. 1G is an enlarged view of the narrow cuts of the plunger of FIG. 1F.

FIG. 1H is an enlarged view of an alternate embodiment of the narrow cuts of the plunger of FIG. 1F.

FIG. 2 is a view of the tube assembly and of the plunger assembly of the present invention, and with the plunger assembly ready to be inserted into the tube assembly.

FIG. 3 is a view of the plunger assembly inserted into the tube assembly, and with the syringe ready for use.

FIG. 4 is a view of the safety syringe of FIG. 3, shown with the break-away plunger screwed into the needle retainer.

FIG. 5 is a view of the safety syringe of FIG. 4, but with the needle and needle retainer withdrawn into the tube, and the plunger being broken off.

FIGS. 5A-5D are the views of FIGS. 2-5 arranged on a single sheet to illustrate the sequence of usage of the safety syringe of the present invention.

### DETAILED DESCRIPTION OF THE INVENTION

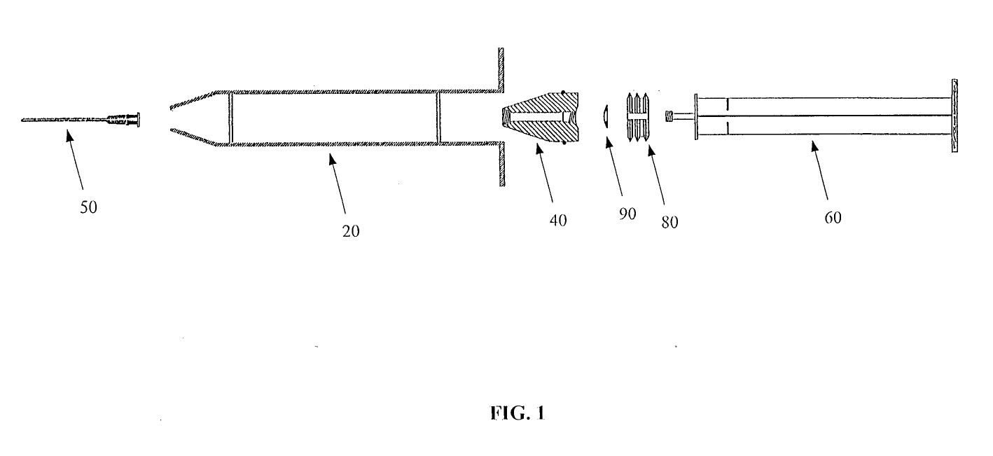

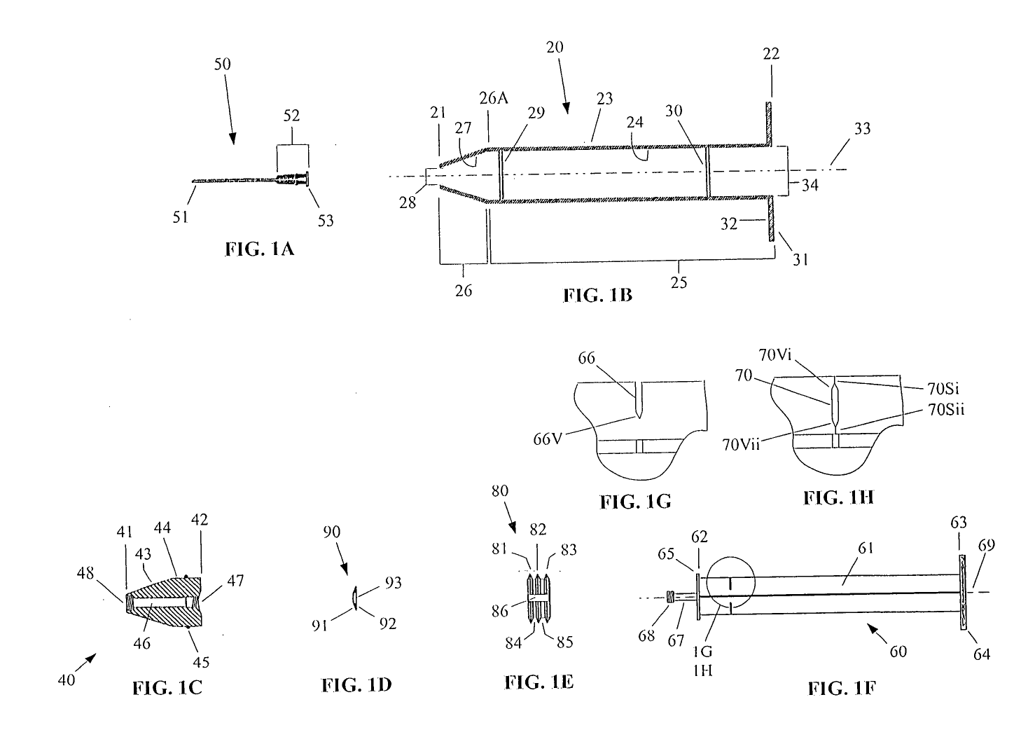

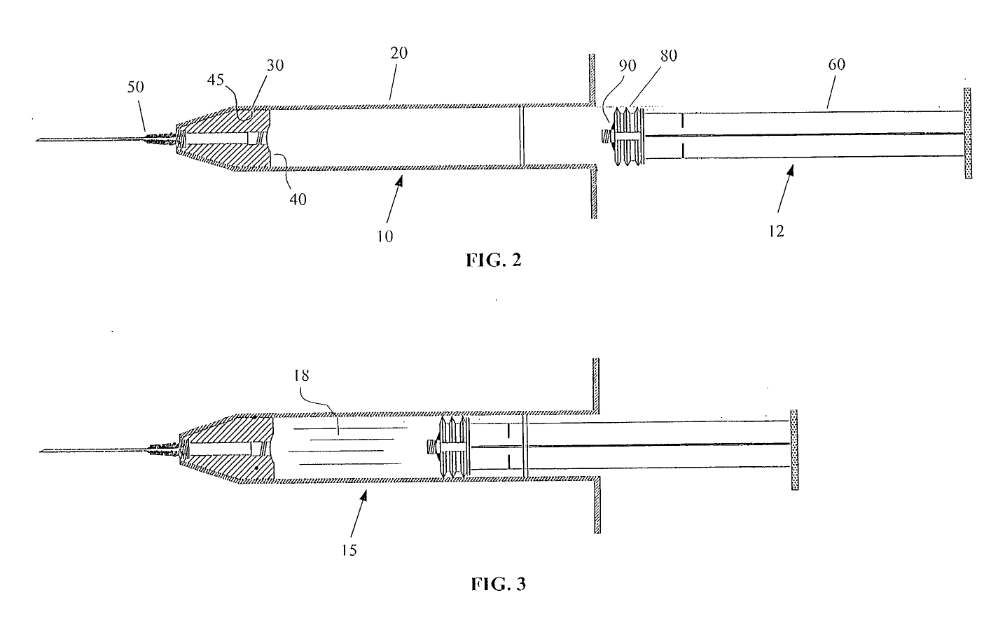

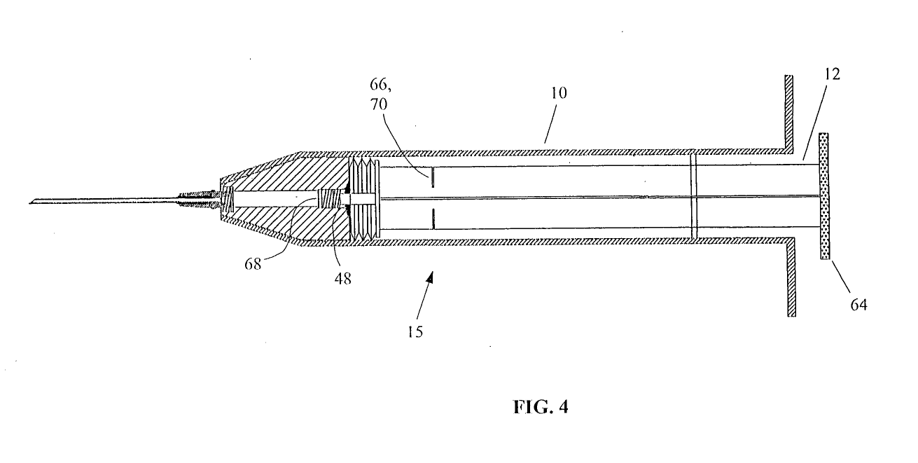

FIG. 1 shows an exploded view of a first embodiment of the present invention, which includes a needle 50, tube 20, needle retainer 40, bellows retainer 90, bellows 80, and a plunger 60. FIG. 2 shows the requisite parts being assembled to produce the tube assembly 10 and the plunger assembly 12, which may be combined to produce the safety syringe 15 of FIG. 3.

As seen in FIG. 1B, the tube 60 may have a first end 21 and a second end 22. The tube 60 may comprise a generally cylindrical tube portion 25 that is hollow, and which has an outside surface 23 and an inside surface 24. The generally cylindrical tube portion 25 may transition at point 26A into a hollow conical frustum portion 26 that has an inside surface 27. An opening 28 may be formed at the first end 21 of tube 60 in the conical frustum portion 26, which may be concentric to the axis 33 of tube portion 25. A disk-shaped flange 31 may be formed at the second end of tube 60 to extend outward and away from the axis 33 of the tube portion 25, leaving a second opening 34 in the tube 20. The flange 31 may have a surface 32 on the tube 25 side of the flange that may be graspable by the user of the syringe 15 (FIG. 3) during its operation to apply pressure to the plunger assembly to cause the injection. The inside surface 24 of the tube portion 25 may have a first annular recess 29 located proximate to transition point 26A of the conical frustum portion 26, and a second annular recess 30 also being located on the inside surface 24, and at a position between the first end 21 and the second end 22 of the tube 20, the functioning of which is discussed hereinafter.

As seen in FIG. 1C, a needle retainer 40 may comprise a cylinder 44 that is formed to be releasably installed through the second end of tube 20 to rest proximate to the first end of the tube 20, and is thus generally sized to be in a close fit with the inside surface 24 of the tube. The cylinder 44 may transition into a conical frustum portion 43, which terminates at a first end 41 of the needle retainer, while the cylinder portion 44 terminates at a second end 42 of the needle retainer 40. The needle retainer 40 may comprise an annular protrusion 45. The dimensions of the annular protrusion 45 of the needle retainer 40 may be set to correspond to those of the first annular recess 29 and second annular recess 30 of the tube 20, as discussed hereinafter. The needle retainer 40 may comprise a bored opening 46 being generally through the center of the cylinder 44 and through the conical frustum 43. The bored opening 46 at the second end 42 of the needle retainer 40 may be tapped to form a series of internal threads 47. Similarly, the bored opening 46 at the first end 41 of the needle retainer 40 may also be tapped to form a series of internal threads 48.

A needle 50 (FIG. 1A) may comprise a base portion 52, from which protrudes a needle portion 51 that is usable to pierce the skin of a patient to accomplish the injection. The base may comprise a ridge 53 that is formed to enable it to be threaded into the needle retainer 40 using the internal threads 48 therein.

The needle 50 may be threadably attached to the needle retainer 40 either prior to insertion of the needle retainer within the tube 20, or thereafter. As seen for the tube assembly 10 in FIG. 2, the needle retainer 40 may be inserted through the opening 34 of the second end 22 of the tube 20, to be advanced toward the first end 21 until the annular protrusion 45 of the needle retainer 40 engages with the annular recess 29 of the tube 20. This engagement of the annular protrusion with the annular recess serves as a detent arrangement in maintaining the needle retainer 40 within the tube 20, until a sufficient force is applied to disengage the needle retainer therefrom, as discussed hereinafter. The location of the annular protrusion 45 along the length of the cylinder 44 of the needle retainer 40 may be such that engagement of the needle retainer protrusion with the tube recess occurs just as the conical frustum 43 of the needle retainer 40 contacts the inside surface 27 of the hollow conical frustum portion 26 of the tube 20. Such contact may serve to prevent the needle retainer 40 from being inadvertently advanced past the point where the annular protrusion rests in the annular recess, to prevent disengagement therefrom either during initial assembly of the tube 10 or during the injection process using the syringe 15 (FIG. 3). The contact may also serve as a secondary sealing means in preventing the escape of the medicant 18 contained within the tube 20 of the syringe 15.

The engagement of the cylinder 44 of the needle retainer 40 with the interior surface 24 of the tube 20 may serve as a primary sealing means for retaining the medicant 18 within the tube. As such, the needle retainer 40 may be made of any suitable material, and may preferably be a material that exhibits some degree of resilience. The tube 20 may be any suitable plastic material, and may preferably be a material that exhibits a fair degree of rigidity to provide stability for the syringe before and during the injection process, as well as for suitably receiving the needle retainer 40 as previously described. In addition, the engagement of the annular protrusion 45 of the needle retainer 40 with the annular recess 29 of the tube 20 may also serve as a sealing means, particularly where the annular protrusion is sized in combination with its resilience for maintaining a high degree of engagement with the annular groove. It should be noted that the resilience of the needle retainer 40 may permit the annular protrusion 45 to be elastically deformed when inserted into the tube 20 during installation therein to form the tube assembly 10. It is also this resilience that provides that the needle retainer 40 is releasably installed within the tube 20, meaning it is also removable therefrom, as discussed hereinafter.

Another key feature of the syringe 15 of the present invention comprises specific components of the break-away plunger assembly 12, as seen in FIG. 2. The break-away plunger assembly 12 may be comprised of a break-away plunger 60, a bellows 80, and a bellows retainer 90. As seen in FIG. 1F, the break-away plunger 60 may comprise an elongated neck region 61 that has a first end 62 and a second end 63. The neck region 61 of plunger 60 may comprise a cross-sectional shape formed by flanges protruding from a central axis 69. There may, in one embodiment, be three equally spaced flanges protruding away from the axis to form a Y-shaped cross-section. In another embodiment, there may be four flanges arranged to form a cruciform-shaped cross-section and thus may generally resemble a plus (+) symbol. A greater numbers of flanges could also be used, but doing so may unnecessarily require greater strength in the caregiver seeking to sever plunger and properly dispose of the syringe. The elongated neck 61 of plunger 60 may have a series of narrow straight cuts 66 in each of the four flanges of the cruciform. These narrow cuts 66 are designed to locally weaken the cruciform shape with respect to the bending strength of the plunger's neck 61. In one embodiment (FIG. 1G), the cuts 66 may begin at the edge of the flange and progress inward towards the axis 69, but stop short therefrom to leave a smaller cruciform cross-section that may support the bellows during the injection process, but be easily severable through bending during the disposal process, as described hereinafter. The straight cut 66 may terminate in a V-shaped notch 66V, which may serve to aid in the severing process. Because these cuts 66 emanating from the edge of the flange make the plunger neck 61 very loose in terms of lateral rigidity, and may tend to reduce the plunger's responsiveness to a doctor or nurse seeking to actuate the plunger, another embodiment seen in FIG. 1H may comprise narrow cut 70 that may not begin at the edge of the flange, and may instead begin just inside the flange edge. Narrow cut 70 may begin with a V-shaped notch 70Vi, and the cut 70 may extend even closer to the axis 69, and may terminate in a second V-shaped notch 70Vii. In this embodiment, each flange may have a hairline slit 70Si that may connect the V-shaped notch 70V to the edge of the flange. In addition, two of the flanges (being two in-line flanges) may also have a hairline slit 70Sii connecting the inner V-shaped notch 70Vii to the start of the wall of the adjacent flanges. It also possible to use hairline slits 70Sii on all four of the flanges. This embodiment, with hairline slits on either two or four flanges, still permits, because the hairline slits are of such a small width, the two sides of plunger neck 61 separated by the slits to bear up upon each other, and thus not present such a loose, spring-like, response to the person manipulating the syringe. At the same time, the slits 70S also greatly aid the user in breaking the plunger at the appropriate time in bending, even where the caregiver does not possess great size or strength.

The neck region 61 may terminate at the first end 62 in a disk 65 that has a radius being sufficient to provide support for the mounting of the bellows 80, while the neck region 61 may terminate at the second end 63 in a disk 64. The disk 64 may be larger than disk 65, as it may thus serve as a knob to be graspable by the caregiver during the injection process or during the break-off procedure, after the syringe has been used and is in need of safe disposal by the caregiver. Protruding out from the disk 65 may be a shaft 67, which may be cylindrical. A portion of the end of the shaft 65 may comprise a cylinder having a slightly larger diameter, and upon which may be a series of external threads 68.

Another key feature of the invention may be found in the sealing means that is secured to the plunger first end, to be useable thereon to seal medication within the tube 20. The sealing means of the current invention is not simply a piston, but instead may comprise a multi-pleated bellows 80. The bellows 80 may preferably comprise three or more pleats, 81, 82, 83, . . . that are connected by flexible troughs 84, 85, . . . , and which form a cylindrical periphery that may engage the inside surface 24 of the tube 20. The multi-pleated bellows 80 serves to ensure adequate sealing even across the annular recess 30 of tube 20, and also ensure sealing integrity during the plunger break-off procedure, because each pleat 81-83 seals independently upon the inside surface 24 of the tube 20, providing a redundancy of sealing against the tube, rather than merely being a single point sealing means, as with the pistons of the prior art. The sealing ability of the bellow 80 is also very adaptive in that each pleat is in itself resilient, while also being flexible with respect to adjacent pleats because of the resilient troughs connecting the pleats together. The bellows 80 may have a cylindrical opening 86 that may permit the bellows to be mounted upon the shaft 67 of the plunger 60. The bellows 80 may be secured thereto using the bellows retainer 90, to create the plunger assembly 12, as seen in FIG. 2.

The bellows retainer 90, as seen in detail in FIG. 1D, may be formed to be comparable to a nut with a threaded opening 93, except that it may have a flat face 92 and an opposite concave surface 91. A pair of tool recesses may be used for threading the bellows retainer 90 onto the shaft 67 of the plunger 60. The threading 68 on the shaft 67 may preferably end at a point that serves to limit the distance that the bellows retainer 90 may be threaded upon the shaft, to prevent any significant compression preloading of the bellows 80, which would tend to undermine its flexible nature, and reduce its sealing capability. In an alternate embodiment, bellow retainer 90 may simply be a C-shaped spring clip that snaps onto shaft 67 to retain the bellow 80 thereon.

Use of the safety syringe herein may be as performed conventionally with most other syringes. The plunger assembly 12 may be inserted through the opening 34 of the second end 22 of tube 20 of the tube assembly 10, as seen in FIGS. 2-3. As there are no interference fit parts that need to be mated herein as with some prior art syringes, assembling of plunger assembly 12 and the tube assembly 10 are completed in a simple manufacturing process. Even the resilient annular protrusion of the needle retainer 40 requires little effort to deform for its insertion into the tube 20. So, the syringe 15 with the component assemblies thus mated per FIG. 3 may be delivered to medical personnel prefilled with medicant, or it may conversely be delivered empty, and the caregiver may manipulate the syringe 15 to draw medicant into the tube, and thereafter dispense it in the usual fashion.

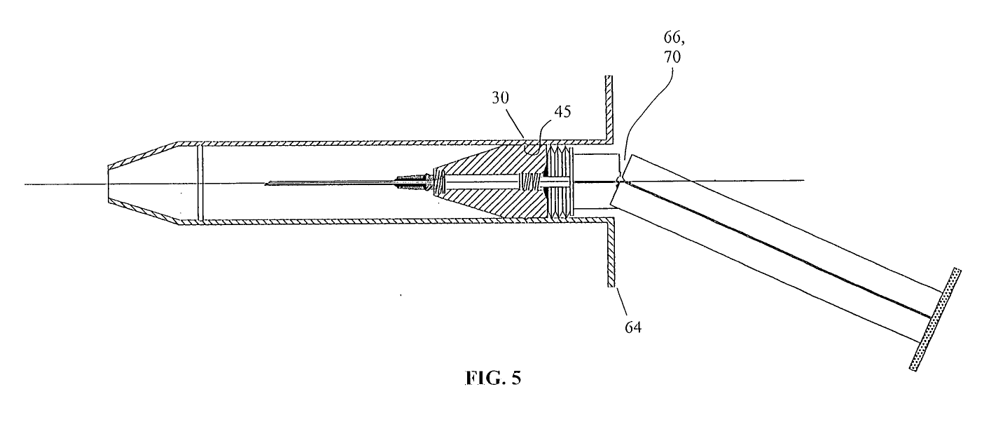

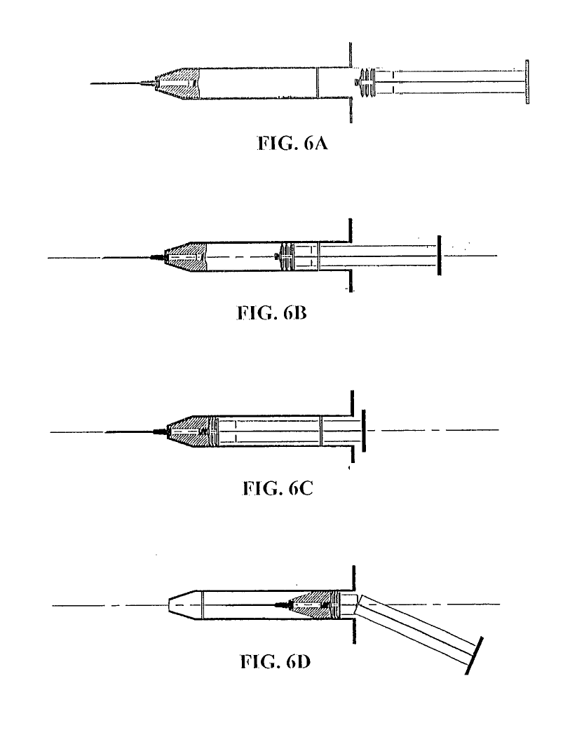

Disposal of the safety syringe 15 of the present invention may proceed as follows. After the injection, which is presumed to be to completion (i.e., the plunger is depressed fully into the tube), the caregiver may use the knob 64 of the plunger 61 to cause the plunger shaft 67 to contact the needle retainer 40 while rotating the plunger assembly 12, so that the external threading 68 of the shaft 67 of the plunger 61 threadably engages the internal threading 47 of the needle retainer 40, as seen in FIG. 4. This threadable engagement may result in some compression of the bellows 80. The user may then grasp the knob 64 and proceed to apply a force relative to the tube 20 to retract the needle 50 and needle retainer 40 into the tube 20 (FIG. 5). Retraction may proceed until the annular protrusion 45 of the needle retainer 40 engages the annular recess 30 of the tube 20, at which time the needle 50 should be safely retracted and be completely within the tube 20. This engagement should occur when the cuts 66/70 are proximate to, or just outside of the tube 20. This engagement serves to aid the user in snapping off the portion of the plunger now protruding beyond the tube 20, by making it easier to simply bend the plunger neck 61 relative to the tube 20. The snapping off of the protruding portion of the plunger is also greatly aided by the narrow cuts 66, and is aided even more so in the embodiment where the cuts 70 with the notches 70Vi/70Vii and hairline slits 70Si/70Sii are used. The multi-pleated bellows 80 also serves, during snapping off of the protruding portion of the plunger, to retain any fluid within the tube 20, despite any deflection of the plunger neck 61 relative to the tube.

The examples and descriptions provided merely illustrate a preferred embodiment of the present invention. Those skilled in the art and having the benefit of the present disclosure will appreciate that further embodiments may be implemented with various changes within the scope of the present invention. Other modifications, substitutions, omissions and changes may be made in the design, size, materials used or proportions, operating conditions, assembly sequence, or arrangement or positioning of elements and members of the preferred embodiment without departing from the spirit of this invention.

What is claimed is:

1. A safety syringe comprising:
a tube, said tube having a first end and a second end;
a needle retainer, said needle retainer being releasably installed in said first end of said tube;
a needle, a portion of a base of said needle being installed in said needle retainer;
a break-away plunger, said plunger having a first end and a second end, said second end comprising a knob for manipulating said plunger;
a sealing means, said sealing means being secured to said plunger first end; and
wherein when said plunger is disposed within said tube using said tube second end and actuated therein, with said sealing means and said needle retainer preventing leakage from said tube, disposal of said safety syringe comprises securing said plunger to said needle retainer to retract said needle into said tube to be retained therein, and breaking off a portion of said second end of said plunger.

2. The safety syringe according to claim 1, wherein said break-away plunger comprises a plunger with four flanges arranged into a cruciform-shaped cross-section; and wherein each of said flanges comprises a narrow cut.

3. The safety syringe according to claim 2, wherein said narrow cut begins at an edge of each of said four flanges.

4. The safety syringe according to claim 2, wherein each of said narrow cuts begins and ends away from a respective edge of each of said four flanges.

5. The safety syringe according to claim 4, wherein said narrow cut begins and ends with a V-shaped notch.

6. The safety syringe according to claim 5, wherein said V-shaped notch at said beginning of each of said cuts connects to a respective edge of each of said flanges using a hairline slit.

7. The safety syringe according to claim 6, wherein said V-shaped notch at said end of each of said cuts connects to a beginning of an adjacent flange using a hairline slit.

8. The safety syringe according to claim 7, wherein said sealing means comprises a bellows.

9. The safety syringe according to claim 8, wherein said bellows comprises three or more pleats, said three or more pleats each sealing independently upon an inside surface of said tube.

10. The safety syringe according to claim 9, wherein said needle retainer being retained within said tube during breaking off of said plunger second end comprises said needle retainer being secured to said tube using a detent.

11. The safety syringe according to claim 10 wherein said detent comprises an annular bearing surface protruding from said needle retainer, and being received by a corresponding recess in said tube.

12. The safety syringe according to claim 11, wherein said needle retainer being releasably installed in said first end of said tube comprises a second detent, said second detent comprising said annular bearing surface of said needle retainer being received in a second corresponding recess located proximate to said tube first end.

13. The safety syringe according to claim 12, wherein said first end of said tube is tapered, with said needle retainer being correspondingly tapered for engagement therebetween, said engagement combining with said detent in providing said releasable installation of said needle retainer in said tube.

14. The safety syringe according to claim 13, wherein said securing of said plunger to said needle retainer during disposal comprises threadably engaging said plunger with said needle retainer.

15. The safety syringe according to claim 14, wherein said needle being secured to said needle retainer comprises threading said needle into said needle retainer.

16. The safety syringe according to claim 15, wherein said sealing means being secured to said plunger first end comprises securing a bellows retainer onto said plunger to retain said bellows thereon.

17. A syringe comprising:
a tube, said tube having a first end and a second end;
a needle retainer, said needle retainer being releasably installed in said first end of said tube;
a needle, a portion of said needle being installed in said needle retainer;
a break-away plunger, said plunger having a first end and a second end, said second end comprising a knob for manipulating said plunger;
a sealing means, said sealing means being secured to said plunger first end; and
wherein when said plunger is disposed within said tube through said tube second end and actuatable therein, disposal of said safety syringe comprises securing said plunger to said needle retainer to thereby retract said needle into said tube to be retained therein, and breaking off a portion of said second end of said plunger.

18. The safety syringe according to claim 17, wherein said break-away plunger comprises a plunger with four flanges arranged into a cruciform-shaped cross-section; and wherein each of said flanges comprises a narrow cut.

19. The safety syringe according to claim 18, wherein said narrow cut begins at an edge of each of said four flanges.

20. The safety syringe according to claim 18, wherein each of said narrow cuts begins and ends away from a respective edge of each of said four flanges.

21. The safety syringe according to claim 20, wherein said narrow cut begins and ends with a V-shaped notch.

22. The safety syringe according to claim 21, wherein said V-shaped notch at said beginning of each of said cuts connects to a respective edge of each of said flanges using a hairline slit.

23. The safety syringe according to claim 22, wherein said V-shaped notch at said end of each of said cuts connects to a beginning of an adjacent flange using a hairline slit.

24. The syringe according to claim 17, wherein said securing of said plunger to said needle retainer during disposal comprises threadably engaging said plunger with said needle retainer.

25. The syringe according to claim 17, wherein said sealing means comprises a bellows.

26. The syringe according to claim 25, wherein said bellows comprises three or more pleats; and wherein each of said three or more pleats independently seal upon said tube.

27. The syringe according to claim 17, wherein said needle retainer being retained within said tube during breaking off of said plunger second end comprises said needle retainer being secured to said tube using a detent.

28. The syringe according to claim 15 wherein said detent comprises an annular bearing surface protruding from said needle retainer being received by a corresponding recess in said tube.

29. The syringe according to claim 17, wherein said needle retainer being releasably installed in said first end of said tube comprises an annular bearing surface of said needle retainer being received in a second corresponding recess located proximate to said tube first end.

30. The syringe according to claim 17, wherein said first end of said tube is tapered, with said needle retainer being correspondingly tapered for engagement therebetween, said engagement combining with said detent in providing said releasable installation of said needle retainer in said tube.

31. The safety syringe according to claim 17, wherein said needle being secured to said needle retainer comprises threading said needle into said needle retainer.

32. The syringe according to claim 17, wherein said sealing means being secured to said plunger first end comprises securing a bellows retainer onto said plunger to retain said bellows thereon.

33. A syringe comprising:
a tube, said tube having a first end and a second end;
a needle retainer, said needle retainer being removably installed in said first end of said tube;
a needle, a portion of said needle being secured to said needle retainer;
a break-away plunger, said plunger having a first end and a second end, said second end comprising a knob for manipulating said plunger;
a sealing means, said sealing means being secured to said plunger first end; and
wherein when said plunger is disposed through said tube second end, disposal of said safety syringe comprises securing said plunger to said needle retainer to thereby retract said needle into said tube to be retained therein, and breaking off a portion of said second end of said plunger.

34. The safety syringe according to claim 33, wherein said break-away plunger comprises a plunger with four flanges arranged into a cruciform-shaped cross-section; and wherein each of said flanges comprises a narrow cut.

35. The safety syringe according to claim 34, wherein said narrow cut begins at an edge of each of said four flanges.

36. The safety syringe according to claim 35, wherein each of said narrow cuts begins and ends away from a respective edge of each of said four flanges; and wherein each of said narrow cuts begins and ends with a V-shaped notch.

37. The safety syringe according to claim 36, wherein said V-shaped notch at said beginning of each of said cuts connects to a respective edge of each of said flanges using a hairline slit.

38. The safety syringe according to claim 37, wherein said V-shaped notch at said end of each of said cuts connects to a beginning of an adjacent flange using a hairline slit.

39. The syringe according to claim 33, wherein said sealing means comprises a bellows, said bellows comprising three or more pleats; and wherein each of said three or more pleats independently seal upon said tube.

40. The syringe according to claim 33, wherein said needle retainer being retained within said tube during break-off of said plunger second end comprises said needle retainer being secured to said tube using a detent; and wherein said detent comprises an annular bearing surface protruding from said needle retainer being received by a corresponding recess in an interior surface of said tube.
